(19) United States
(12) Patent Application Publication
(10) Pub. No.: US 2021/0346638 A1
(43) Pub. Date: Nov. 11, 2021

(54) Shared Manifold Ventilator and Method of Use

(71) Applicant: Roger W Faulkner, Davidson, NC (US)

(72) Inventor: Roger W Faulkner, Davidson, NC (US)

(21) Appl. No.: 17/317,871

(22) Filed: May 11, 2021

Related U.S. Application Data
(60) Provisional application No. 63/022,749, filed on May 11, 2020.

(51) Int. Cl. A61M 16/20; A61M 16/08

(52) U.S. Cl. CPC A61M 16/202; A61M 16/0875; A61M 2205/84; A61M 2016/0027; A61M 2202/0266; A61M 2205/3331; A61M 2202/0208

(57) ABSTRACT

The Shared Manifold Ventilator uses low pressure breathing gas manifolds to interface directly with patients in a hospital ward, through solenoid valves in such a way that it achieves lower cost per patient ventilated than prior art methods while still allowing full control of the breathing cycle and oxygen concentration for each individual patient.

### CROSS-REFERENCE TO PRIOR FILED APPLICATIONS

This application claims priority to an earlier-filed provisional application, serial no. 63/022,749 filed in the United States Patent Office on May 11, 2020, which is herein incorporated by reference in its entirety.

### TECHNICAL FIELD

The present disclosure generally relates to an apparatus for providing breathing gas to a patient. More specifically, this disclosure relates to an apparatus capable of providing breathing gas to a plurality of patients. This disclosure also relates to the operation of providing breathing gas to a plurality of patients through the use of an apparatus with a shared manifold. This disclosure further relates to a ventilator system that is comprised of solenoid valves and, optionally, a balloon accumulator.

### BACKGROUND ART

During the recent COVID-19 pandemic there were multiple proposals and actual applications for shared manifold ventilators in which all the patients on the manifold had to have the same breathing cycle. These designs share the output from a conventional ventilator between multiple patients. For example, these problems are discussed in:—Laffey, John G et al. “Supporting more than one patient with a single mechanical ventilator: useful last resort or unjustifiable risk?” British Journal of Anaesthesia vol. 125,3 (2020): 247-250. doi:10.1016/j.bja.2020.05.029—, hereby incorporated by reference in its entirety.

In the prior art designs to share a single ventilator with multiple patients, pressure changes in the entire manifold actuate breathing of all the patients on the manifold. The oxygen to air ratio is fixed for all the patients on the manifold. The inhalation pressure is fixed for all the patients on the manifold. The inhalation time is the same for all patients on the manifold. PEEP is usually not individually adjustable for each patient. Such a design is not suitable for five or more patients, and is not desirable for even two patients.

To illustrate the prior art, FIG. 2 shows a typical prior art ventilator for three patients 71. Each patient interfaces with a common breathing gas manifold 62 through a virus-capable filter 63. The exhalation cycle begins when solenoid 67 opens; the exhausting gas goes through virus capable filter 64 and exhausts through the controllable pressure relief valve 68. The breathing gas supply is created by mixing air from connection 87 and oxygen from connection 77 in the breathing gas mixing device 88. Oxygen is supplied from a high-pressure tank 72 through pressure regulator 73 and through the humidity and temperature adjustment device 74; following this, the oxygen goes through a one-way valve 75 and enters the connecting tube 77 to the oxygen/air mixing device 88. Air is supplied by blower 82, through one-way valve 83, and then through virus-capable filter 84, after which it enters the humidity/temperature adjustment device 85, then passes through a second one-way valve 86 after which it enters the connecting pipe 87 to the breathing gas mixing device.

There exists a need and a desire for an apparatus capable of providing a breathing gas in a consistent fashion to a plurality of patients. More particularly there is a need and desire for an apparatus that is capable of providing a breathing gas in a consistent fashion to five or more patients. More particularly there is a need and desire for an apparatus that is capable of providing a breathing gas in a consistent fashion to an entire ward of respiration-compromised patients.

Presented herein is an apparatus comprising a single manifold and a plurality of solenoids that is capable of providing a breathing gas in a consistent fashion to plurality patients, including five or more patients. The apparatus presented here can be configured to provide breathing gas in a consistent fashion to an entire ward of respiration—compromised patients.

### SUMMARY OF THE INVENTION

At its core the invention is a manifold ventilator capable of servicing multiple patients comprising, at least one breathing gas supply manifold; a plurality of lengths of gas exchange tubing; at least two solenoid valves and at least two breathing tubes. Furthermore, each solenoid valve has a gas inlet connection and a gas outlet connection. Also for at least two solenoid valves, the gas inlet connection is connected to the breathing gas supply manifold by a length of gas exchange tubing and the gas outlet connection is connected to a breathing tube. Additionally, when at least one of the solenoids is in the open position there is an isolated passageway for breathing gas from the breathing gas supply manifold through the open solenoid to the breathing tube.

In a preferred embodiment, the manifold ventilator additionally comprises at least two breathing gas supply manifolds, wherein the breathing gas of at least two breathing gas supply manifolds can be mixed prior to the entry of the gases into at least one breathing tube. In a more preferred embodiment of the invention, data from a pulse oximeter is used to adjust the opening time of an oxygen solenoid and an air solenoid providing breathing gas to a patient. In another more preferred embodiment of the invention wherein for at least one patient receiving a breathing gas, the solenoid valve in line with the oxygen manifold is opened and closed before the solenoid valve in line with the nitrogen manifold is opened and closed, in which the oxygen going to the patient is controlled by the relative open time of the solenoid valve in line with the oxygen manifold compared to be an open time of the solenoid valve in line with the nitrogen manifold. In an even more preferred embodiment, the relative opening time of the solenoid valve in line with the oxygen-containing manifold compared to the opening time of the solenoid valve in line with the nitrogen-containing manifold is controlled to achieve a target oxygenation levels measured by a sensor worn by the patient. In an even more preferred embodiment of the invention, breathing gas is simultaneously supplied to at least three patients, wherein at least two of these patients receive a breathing gas with a different level of oxygen concentration.

In a more preferred embodiment of the invention, breathing gas is supplied to three or more patients. In an even more preferred embodiment of the invention, breathing gas is supplied to at least one patient that has been diagnosed as infected with COVID-19 virus. In a still further preferred embodiment breathing gas is simultaneously supplied to at least three patients that have been diagnosed as infected with COVID-19 virus.

In a more preferred embodiment, at least one breathing gas supply manifold supplies a breathing gas that is at least 50% oxygen. In another preferred embodiment, at least one breathing gas supply manifold supplies a breathing gas that is at least 50% nitrogen.

In another preferred embodiment, the ventilator comprises at least one exhaust manifold capable of maintaining a positive end expiratory pressure (PEEP). In a more preferred version, the ventilator comprises at least one vacuum accumulator connected to the exhaust manifold.

In yet another preferred embodiment, the ventilator comprises at least two elastomeric balloon pressure accumulators attached to at least two breathing tubes. In a more preferred embodiment of the invention, a pressure sensor senses the pressure in one or more bedside balloon accumulators so as to control the inhalation pressure for that individual patient. In another more preferred embodiment, an oxygen sensor is linked to a bedside balloon accumulator and provides oxygen concentration data to the control system which is used to control the opening time of an oxygen solenoid and linking the bedside balloon accumulator to the patient manifold

In a similar preferred embodiment, the ventilator comprises at least one balloon accumulator attached to at least one of the breathing gas supply manifolds.

In still another preferred embodiment, the ventilator comprises at least one sensor positioned on the air manifold; said sensor being electronically linked to a control system for at least one solenoid valve to exhaust air from the air manifold in order to accurately maintain a target pressure therein.

In another embodiment the invention as described above can be use as a method of treating the respiratory distress symptoms of a patient diagnosed with COVID-19 virus. It is well-known that exposure to COVID-19 virus can be determined by clinical tests. It is well-known that a skilled medical practitioner can recognize and diagnose symptoms of respiratory distress that a well-established to be typical of patients exposed to COVID-19 virus. Obviously the invention can be used to treat any patient with respiratory distress. In certain embodiment the invention can be used to supply specialized breathing gas to healthy patients (e.g. to induce acclimation to low oxygen environments). Similarly, the invention can be used to treat or prevent conditions associated with exposure to unusual environments; for instance the invention can be used by divers to supply breathing gases at higher pressure or in exotic mixtures of oxygen and or other gases (for instance, helium) to treat or prevent decompression sickness (DCS) or Caisson disease (the bends).

### BRIEF DESCRIPTION OF DRAWINGS

FIG. 1 is a drawing that depicts a shared manifold (X, Y), with multiple solenoids (Z) as embodied in Example 1; also shown are balloon accumulators (A, B, C) on both the air (X)and oxygen manifolds(Y);

FIG. 2 is a drawing that illustrates an example of a previously disclosed manifold ventilator which is found in prior art and described in Example 2 (accordingly, this drawing is marked “Prior Art”).

FIG. 3 is a drawing that depicts an alternative layout for the patient interface as described in Example 3.

FIG. 4 is a drawing that depicts a design in which the manifolds loop around the hospital ward as described in Example 4.

### DISCLOSURE OF INVENTION

### Definitions

The term “air manifold”, as used herein, refers to the shared breathing gas manifold containing either normal atmospheric air any non-toxic breathing gas mixture with a relatively lower oxygen content than the oxygen manifold (if present).

The term “oxygen manifold”, as used herein, refers to the shared breathing gas manifold containing which supplies a gas containing oxygen to the patients. The oxygen manifold can be distinguished from the air manifold by virtue of the gas within the oxygen manifold is relatively higher in oxygen content than the air manifold. Any non-toxic grade oxygen is suitable for use within the oxygen manifold, for instance, medical-grade oxygen or welding-grade oxygen (which contains about 5% argon).

The term “patient manifold”, as used herein, refers to the section of tubing that is in communication with a solenoid valve linking to the oxygen manifold, a solenoid valve linking to the air manifold and the solenoid valve linking to the exhaust.

The term “breathing gas”, as used herein, refers to either the contents of the air manifold or the oxygen manifold.

The term “PEEP,” as used herein, refers to positive end expiratory pressure. This term is used to describe the minimum pressure at the end of exhalation.

The term “solenoid valve”, as used herein, refers to a valve that is actuated by electrical means. The valve portion of the solenoid valve has at least a fully open position and at least a fully closed position, the position of which is actuated by electrical means such as an electric motor drive or by an electromagnetic helical coil. The solenoid valve as used herein can also be fluid assisted such as a pneumatic or hydraulic valve. A solenoid valve of the described invention has a gas inlet connection and gas outlet connection. When the solenoid valve is in the fully open position there is an unobstructed pathway for gas to flow from the inlet connection through the valve to the gas outlet connection. The terms “solenoid valve” and “solenoid” are used interchangeably herein.

The drawings are marked “FIG.” followed by a numeral. As used herein the abbreviation “FIG.” and the word “figure” are synonymous and used interchangeably.

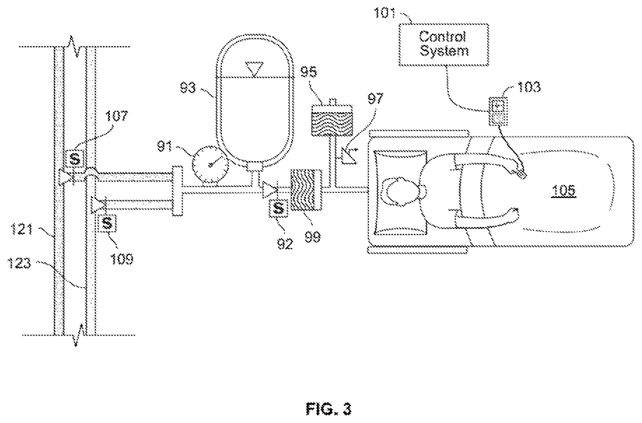

The master controller in some embodiments of the manifold is abbreviated “CS”, see FIG. 3 of the drawings.

The terms “closed loop” or “closed passageway” are used herein to indicate a passage for gas through pipes that is isolated from the air in the environment in which the manifold in housed. The terms “closed loop” is synonymous with “isolated loop”; likewise the terms “closed passageway” is synonymous with “isolated passageway”

As used herein the term “inch” means the English unit of measure for distance. As used herein, a single inch is defined as 2.54 cm. The unit inch is often abbreviated by the symbol —“— referred to as double quotation marks or double prime marks.

As used herein the abbreviation “Pa” indicates pascal, the SI unit for pressure is the pascal. The unit is understood to equal to one newton per square meter (N/m2, or kg·m−1·s−2). Pressure is often amplified and reduced by SI prefixes, such as “kPa” and “mPa”. Background literature may use other non-SI units (and abbreviations) for pressure such as Standard Atmosphere (atm)”, inches of mercury (in HG or HG), centimeters of mercury (cm HG), millimeters of mercury (mm HG or—historically—Torr),—modern—Torr (Torr or mTorr) and pound per inch (psi). Within the industry, it is well understood how to convert these pressures defined by these units to the SI-standard unit, pascal.

### DETAILED DESCRIPTION OF THE INVENTION

The subject of this disclosure, the shared manifold ventilator, relies on shared breathing gas manifolds that can extend all-around a shared ventilation ward. These manifolds may be held at an appropriate temperature, pressure and humidity for direct use for ventilating patients, without any mechanical pressure change devices between the manifolds and the patient.

Every patient served by the shared manifold ventilator is connected to at least one shared manifold containing breathing gas, which is held at an appropriate pressure for ventilation without any mechanical pressure drop mechanism between the manifold and the patient. There are in most cases an oxygen manifold and an air manifold, which are both at nearly the same pressure, and which are suitable for direct interface with the patient for inhalation. This common breathing gas manifold pressure would typically be no more than 5000 pascals above atmospheric pressure. The hardware and sensors required for providing breathing gas at a prescribed pressure, temperature and humidity through these manifolds is a shared expense over as many patients as are served by the manifolds.

There may also be a means to apply positive end expiratory pressure (PEEP). PEEP can be controlled at each patient's bedside by a variable pressure relief valve. The exhausted gas could go into the atmosphere of the hospital ward after being filtered, or it could go into a shared exhaust manifold.

The oxygen and air manifolds ideally are at a pressure appropriate for direct ventilation of a patient, or with a slight pressure modification by use of a bedside pressure accumulator. Because there is no need to step down the pressure at each patient's bedside by means of a mechanical pressure regulator, the device can be simplified. This design also removes the need for accurately pre-mixing oxygen and air for each patient. This reduces cost and also simplifies maintenance.

Maintaining a very low-pressure differential throughout the manifolds may require the manifold diameters to be larger than the pipes that are normally used to distribute oxygen in a hospital for example. Since resistance to flow in a pipe scales with the inverse fourth power of the diameter, typically the air and oxygen manifold pipes do not need to be bigger than 10 cm inside diameter to guarantee no more than +/−5% change of pressure inside the manifold from the setpoint pressure, but manifold pipe diameters larger than this might be required for very large ventilation wards.

Because of the low pressures involved, no more than 5% above normal atmospheric pressure, the solenoid valves for use on this device may be much less expensive when compared to the solenoid valves used to control compressed air or oxygen in typical industrial gas handling equipment, or in background hospital ventilators described in the literature. Electronic pinch valves such as those mentioned in U.S. Pat. No. 4,548,382A—hereby incorporated by reference in its entirety—are desirable for use as the solenoid valves of this invention.

Each patient is linked to each manifold via a solenoid valve. In line with each solenoid valve, there may, optionally—and preferably—, be a one-way valve and—preferably—also a virus—capable filter. Ventilation of each patient is made simple in that the breathing gas supply is controlled by two solenoid valves and the exhaust is controlled by a third solenoid valve. Preferably, there is also a controllable pressure relief valve on the outward side from the patient after the exhaust solenoid valve to provide PEEP.

A further desirable modification of the invention entails inflatable balloons which work as gas pressure accumulators at the patient bedside, in which oxygen and air are sequentially added to said balloon accumulators through the solenoid valves connecting to the oxygen and air manifolds. In this scenario, one additional solenoid valve between the patient and the balloon accumulator at the bedside of each patient is needed. Using these balloon accumulators enables individualized pressure control for each patient, while simultaneously providing a visible indication that the device is working, which is not based on electronics.

A desirable means of providing individualized PEEP for each patient is via a variable and/or controllable pressure relief valve beside each patient. PEEP is not desirable for all patients, however for those in respiratory distress, a positive PEEP pressure is highly desirable. Any such pressure relief valve would ideally be triggered based on the difference between the pressure entering the valve and the local atmospheric pressure, rather than the pressure differential across the valve since the virus capable filter or the exhaust manifold will impose a variable pressure differential while exhaust gas is flowing.

Any air exhausting directly to the hospital ward should be sterilized and/or filtered. Alternatively, all the exhaled gas can exit the hospital ward through an exhaust manifold.

By controlling the opening and closing times of the oxygen solenoid and the air solenoid, any oxygen level can be supplied for each individual patient, this includes but is not limited to industry-standard, 21% oxygen up to pure oxygen, and includes values in between such as 30%, 40%, 50% 75% and 90% partial press of oxygen. If it were medically desirable to have lower than 21% oxygen, for instance, to induce acclimation, the system could be configured to supply 10%, 12%, 15, 17%, 18% and 20% partial press of oxygen or values in between. In the case that a balloon accumulator is used, one can also control the inhalation pressure for each individual patient. By incorporating pulse oximeters for each patient, the oxygen ratio can be automatically adjusted to meet targets for blood oxygenation.

### MANIFOLD VENTILATOR

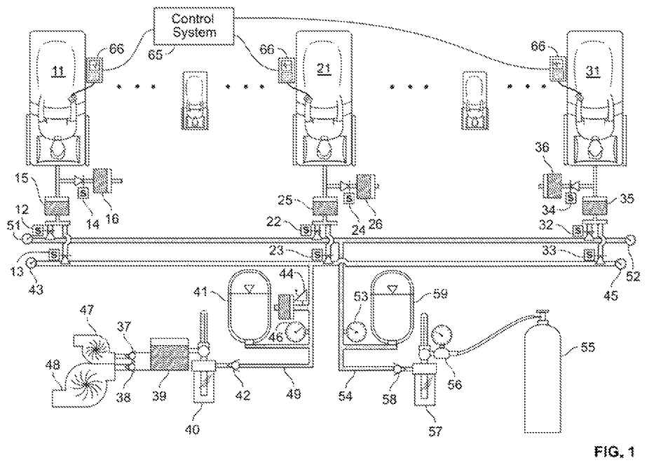

A manifold ventilator provides clean, oxygenated breathing gas to a plurality of patients while maintaining regulated PEEP for each patient. In the less complex form, the manifold lacks a balloon accumulator. The version is described in pragmatic and functional terms in Example 1. Below this embodiment of the invention is described elemental parts; FIG. 1 is referred to.

In the manifold ventilator that is not equipped with a balloon accumulator, the pressure in both the air manifold and the oxygen manifold would normally be well above the local atmospheric pressure, preferably between 1500 to 3500 pascals above local atmospheric pressure. For a typical patient in this case, the oxygen solenoid valve opens for a time that would be between 0.01 to 3 seconds, and then the air valve opens up until the end of the inhalation cycle, which would typically be between 1 to 3 seconds. In some embodiments, there can be engineered a hold time before the beginning of the exhalation cycle. In this scenario, the oxygen concentration in the inhaled gas will change during the course of the inhalation. By allowing the oxygenated breathing gas to enter into the patient's lungs first, and then blowing it further into the lungs with air, wastage of oxygen will be minimized. This is particularly important for clinics that have a limited oxygen supply.

FIG. 1 shows two manifolds. The oxygen manifold 54 is supplied with oxygen from an oxygen tank 55, through a pressure reduction regulator 56, and then has its humidity and temperature adjusted by regulator 57 before it enters the oxygen manifold. The air manifold is powered by two blowers 47 and 48, which blow air through one-way valves 37 and 38, a HEPA filter 39, followed by a humidity/temperature control device 40. Humidity and temperature-controlled air enters the air manifold near manifold balloon accumulator 41 in the middle of the manifold. Similarly, humidified oxygen enters the oxygen manifold near the center point, right beside an oxygen manifold balloon accumulator 59 to dampen pressure fluctuations in the oxygen manifold.

FIG. 1 shows a linear manifold with three patient take-off points (11, 21 and 31); patient #1 11 is at the left end of the manifold, patient #10 21 is in the middle of the manifold, and patient #20 31 is at the right end of the manifold. Beside each patient is a bedside communications, data and control hub 66 which are able to control the solenoid valves used for each patient. This bedside control unit also interfaces with the master controller (Marked ‘CS’ in FIG. 3) 65 which would normally be located at the nursing station for the hospital ward.

There are three pressure sensors for each of the manifolds. On the air manifold, the pressure is sensed on the left-hand side 49 of the manifold by sensor 41, pressure sensor 42 senses the pressure at the right end of the manifold, and pressure sensor 43 and 45 are located at the left and right ends of the manifold, pressure sensor 46 is located near the middle of the manifold. Data from Sensors 43 and 45 is used to control exhaust valve 44 which is located next to the balloon accumulator at the midpoint of the air manifold.

On the oxygen manifold, pressurized oxygen is supplied by oxygen tank 55, through pressure regulator 56, after which the low-pressure oxygen goes through the humidity/temperature regulator 57. After the low-pressure oxygen is humidified, it goes through one-way valve 58 after which it enters a zone in communication with the oxygen balloon accumulator 59. Pressure is sensed on the left-hand side of the manifold by sensor 51, pressure sensor 52 senses the pressure at the right end of the manifold, and pressure sensor 53 is located near the middle of the manifold. Pressure sensor 53 is used to monitor manifold pressure and/or to control manifold pressure by adjusting the pressure regulator 56

Patient #1 (11) is served by a breathing gas mixture comprised of oxygen coming through solenoid 12 and/or air coming through solenoid 13 after which the breathing gas mixture goes through inlet filter 15. Between 15 and the patient 11 is a section of tubing that also attaches to the exhaust solenoid 14 followed by exhaust into the hospital ward through exhaust filter 16.

Patient #10 21 is served by a breathing gas mixture comprised of oxygen coming through solenoid 22 and/or air coming through solenoid 23 after which the breathing gas mixture goes through inlet filter 25. Between 25 and the patient 21 is a section of tubing that also attaches to the exhaust solenoid 24 followed by exhaust into the hospital ward through exhaust filter 26.

Patient #20 31 is served by a breathing gas mixture comprised of oxygen coming through solenoid 32 and/or air coming through solenoid 33 after which the breathing gas mixture goes through inlet filter 35. Between 35 and the patient 31 is a section of tubing that also attaches to the exhaust solenoid 34 followed by exhaust into the hospital ward through exhaust filter 36.

Data from the various sensors is sent into a control system (not shown in FIG. 1). Said control system receives all sensor inputs and triggers the solenoids and blowers. The actual connections between the control system and the sensors and solenoids may be wired connections, Wi-Fi or Bluetooth connections, or fiber-optic connections.

### BALLOON ACCUMULATOR

Balloon accumulators for use with the disclosed invention are any type of reservoir capable of storing excess gas from the ventilator system when the local volume and/or local pressure increases over baseline volume and/or pressure. Typically balloon accumulators feature a bladder constructed from elastic material, preferably a rubber material, into which excess gas can be stored and from which stored gas can be retrieved.

Balloon accumulators of the disclosed invention can be free bladders without housing. Alternatively, the bladder may be contained within a housing for protection. Certain embodiments feature a balloon accumulator in which the bladder is contained in a rigid housing and surrounded by another fluid. The surrounded fluid could be an incompressible fluid such as water, or the surrounding fluid could be a pressured gas, such a gas substantially composed of nitrogen.

Balloon accumulators for this application should be very resistant to aging and oxidation. Balloons accumulators selected for use with a particular embodiment of a manifold ventilator ought to have a pressure rating that exceeds the maximum expected operating pressure for the gases within the pipes to which the balloon accumulators are attached.

### MANIFOLD VENTILATOR WITH AFFIXED BALLOON ACCUMULATOR

In the case wherein the manifold ventilator is coupled to a balloon accumulator holding the breathing gas mixture at the inlet tube for each patient, the cycle will be slightly different from that of the ventilator without balloon accumulators. This embodiment—a manifold ventilator coupled to a balloon accumulator—and will be more tolerant of higher manifold pressure, since the balloon accumulator can be used to control the inhalation pressure. In this scenario, the oxygen content of the breathing gas is controlled by the timing of the oxygen and air valve openings into the accumulator balloon. These solenoid valve openings and closings do not need to be synchronized to the breathing cycle, which will be controlled by a separate solenoid valve between the balloon accumulator and the patient, as shown in FIG. 3.

FIG. 3 shows an individual takeoff station for a patient 105 and includes a balloon accumulator 93 for mixing air and oxygen. Oxygen enters the balloon through solenoid valve 107, which attaches to the oxygen manifold 121. Air enters the balloon through solenoid valve 109, which attaches to the air manifold 123. Between balloon accumulator 93 and patient 105 is a solenoid valve 92 and a virus capable filter 99. FIG. 3 shows an exhaust solenoid 98 and a pressure relief valve 97 so that the exhaust goes through a virus removal device 95 before exhausting to the hospital ward. Said virus removal device could be either a virus-capable filter or a sterilization zone involving ultraviolet diodes capable of sterilizing the air even if the filters fail. Pressure in the balloon accumulator is measured by 91 and the information is passed back to control systems 101 and 103. Bedside control system 103 is capable of receiving instructions from the master control system 101 but is also capable of independently controlling solenoid 107, 108, and 98. An optional oxygen sensor (not shown) may be inside the balloon accumulator 93.

FIG. 3 shows a bedside control system 103 which is normally slaved to control system 101 which receives input from sensors and creates output to control solenoid valves or devices such as temperature and humidity control devices, blowers, oxygen pressure regulators, and/or variable flow restriction devices. More maximum safety, the bedside control module 103 should be able to operate independently and should have built-in redundancy.

The oxygen and air manifold pressures should not be so high as to be capable of bursting the balloon accumulator but are not otherwise limited in this case. In some embodiments, the gas supply manifold pressures can even be higher than the burst pressure of the bedside balloon accumulators, provided safety measures are taken so that the pressure is reduced before the breathing gas reaches any of the balloon accumulators. This super-high embodiment—while possible—is not practical for most users as the additional safety issues—generally—outweigh the advantages of operating the manifold at such super-high pressures. It is generally desirable to keep the manifold pressure below that which could burst the bedside balloon accumulators. The balloon accumulators, by enabling higher manifold pressures also enable higher pressure drops in the manifold and therefore smaller manifold pipe diameters.

Balloon accumulators have a significant advantage compared to rigid gas pressure accumulators. When additional gas is put into a balloon accumulator, gas pressure changes only slightly because the pressure goes mostly to expanding the balloon diameter mostly, rather than to increase the pressure inside the balloon. Balloon accumulators for this application should be very resistant to aging and oxidation. Balloons accumulators selected for use with a particular embodiment of a manifold ventilator ought to have a pressure rating that exceeds the maximum expected operating pressure for the gases within the pipes to which the balloon accumulators are attached.

For this embodiment, the exhalation cycle is typically between 1-3 seconds. The exhalation cycle is controlled by the exhaust solenoid which is beside each patient. PEEP can be maintained by a pressure relief valve on the other side of the exhaust solenoid from the patient. Depending on the configuration of the manifold ventilator and the needs of the facility operating it, the exhaled gas can go into an optional exhaust manifold that vents to an area outside of the facility, or it can vent through a virus capable filter or through a device to kill the virus before entering the atmosphere inside the hospital. It is important to remove or kill any aerosolized virus particles before the exhaled gas can be vented inside the facility.

In one embodiment, a valve controls the pressure in the air supply manifold by venting air from the manifold to control the pressure. In this scenario, air is flowing into the air manifold to a greater extent than the minimum airflow required for ventilating the patients served by the air manifold. Another way to control the pressure in the air manifold is to have a variable speed drive for the blower which is pressurizing the air manifold. In either case, it is desirable to have a balloon accumulator at the midpoint of the manifold and to have a pressure sensor located near the mouth of the balloon accumulator that controls the blower fan and/or the exhaust valve to control the midpoint air manifold pressure. The best location to introduce air or oxygen to the air manifold and oxygen to the oxygen manifold is right beside the manifold balloon accumulators, at the midpoint of the breathing gas supply manifolds.

A low-cost means to control the pressure in the air manifold is with a blower and a pressure relief valve. It is desirable to select the air manifold pipe diameter so that the manifold differential pressure from local atmospheric pressure varies by no more than 5% from the setpoint gauge pressure throughout the air supply manifold.

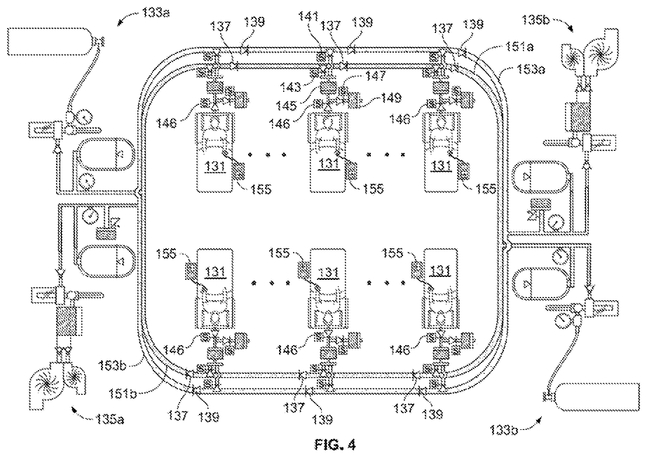

Both the air manifold and the oxygen manifold can be equipped with balloon accumulators to minimize pressure fluctuations during the opening and closing of valves that serve patients on the manifolds. It is within the scope of this invention and also desirable that there is more than one point of pressure introduction into the manifolds for air and oxygen, and more than one manifold balloon accumulator for each breathing gas manifold, as shown in FIG. 4.

FIG. 4 shows an example of manifolds that loop completely around the hospital ward, serving multiple patients 131 and with two different pressurization sources for both the oxygen 133a, 133b and air manifolds 135a, 135b. There closed passage for oxygen breathing gas 151a, 151b and a closed passage for air breathing gas 153a, 153b. There are valves between each next-neighbor set of patients, these sectionalizing valves are 137 for oxygen and 139 for air so that the loop manifold can be sectionalized. The patients 131 receive oxygen through solenoid valve 143 followed by air that comes through solenoid valve 141; the air and oxygen pass through a filter 145 before going to the patient 131. Exhaust solenoid valve 146 opens during the exhalation cycle which is triggered by control system 155 after the exhaust gas flows through 146 it passes through adjustable pressure relief valve 147 which may be manually controlled or it can be controlled by 155. The bedside control module 155 will, in most cases, receive instructions from the master controller at the nursing station.

The oxygen manifold pipe, and any balloon accumulator that might see pure oxygen must be compatible with exposure to highly concentrated oxygen. At 101 kpa, or one atmosphere, oxygen pressure, practically any polymer is suitable for use with the invention. Metal pipes and fluoropolymers are also suitable for use with the invention, some local regulations may require the use of these materials. Any configuration of the disclosed invention ought to consider and comply with all the regulations of the jurisdiction in which the invention is located and where it will be used. It is understood that PVC pipe will be safe for this application. PVC pipe is the preferred material for use with the invention provided the use of PVC pipe is permitted in the local of use. Any balloon accumulator that can withstand the anticipated pressures of the manifold ventilator and is permitted for use in the local jurisdiction can be used with the disclosed invention. It is preferred that the balloon accumulators are constructed from oxidation-resistant elastomers with saturated backbones such as EPDM, thermoplastic elastomers, or silicone.

### SOLENOID VALVE

A solenoid valve is a valve that can be actuated—at least in part—by electronic means. The valve portion of the solenoid valve has at least a fully open position and at least a fully closed position, the position of which is actuated by electrical means such as an electric motor drive or by an electromagnetic helical coil. The solenoid valve as used with the described invention can also be fluid assisted such as a pneumatic or hydraulic valve. A solenoid valve of the described invention has a gas inlet connection and gas outlet connection. When the solenoid valve is in the fully open position there is an unobstructed pathway for gas to flow from the inlet connection through the valve to the gas outlet connection. Solenoid valves can be actuated by a wired electric signal or electric charge from a circuit. Alternatively, solenoid valves can be operated with a wireless signal, such as but not limited to microwave signal, radio signal (including, but not limited to WiFi, Bluetooth), sound, and light (including IR).

Solenoid valves are particularly useful with the present invention because they offer fast and safe switching, high-reliability and long service life. Such qualities combined with electric control permits for high performance, reliability and responsiveness that enhances the capability of the shared manifold ventilator of the present disclosure to deliver breathing gases to a plurality of patients.

The present invention can be constructed with a wide variety of solenoid valves. Most embodiments use two-port valves; however, it envisioned that certain embodiments could use configurations of solenoid valves with more than two ports. In certain embodiments of the invention, a solenoid valve is implemented with a spring mechanism to hold the valve in an open position until is actuated to be closed, i.e. normally open valves; likewise, other embodiments utilize a solenoid valve comprising a spring to hold the valve closed until actuated to be opened, i.e. normally closed valves.

Solenoid valves for use with the present invention can be constructed from, but not limited to, brass, stainless steel, aluminum, and plastic. The seals of the solenoid valves for use with the are typically, but not limited to, metal or rubber; preferably the seals used in the present invention are FKM, EPDM and/or PTFE.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

Example 1 is illustrated in FIG. 1. In this case, 20 patients in a hospital ward are served by an oxygen manifold and an air manifold, both of which are comprised of plastic pipes with an inside diameter of 10 cm. The oxygen and air manifolds have a gas pressure supply at only one point, at the midpoint of the manifold. Oxygen is supplied through a pressure regulator, followed by a humidifier which also controls temperature. The humidity of the oxygen and air would typically be adjusted to approximately 65% relative humidity. The air manifold in Example 1 is powered by two blowers, each of which is linked to the air manifold through a one-way valve. Airflow into the air manifold can be supplied by one small blower, one larger blower with twice the capacity of the small blower, or both blowers operating together.

In FIG. 1 and Example 1, the two blowers are sized so that the second larger blower has twice the capacity of the smaller blower. The smaller blower can serve up to seven patients, the second larger blower can by itself serve up to 14 patients. If both blowers are on simultaneously, they can serve 21 patients on the air manifold This setup enables three air output levels.

As the number of patients on the manifold increases, at first only the small blower will be used, then as the number of patients increases beyond the capacity of the small blower, the small blower is turned off and the larger blower takes the load. When the manifold serves more than about 14 patients, both blowers are on at the same time. This methodology creates a low-cost means to pressurize the air manifold at relatively low levels of energy consumption. In this case, the air manifold pressure level is controlled by means of exhausting excess air from the manifold blower just downstream from the air manifold balloon accumulator. The controller which controls the exhaust valve receives its input from a pressure sensor located near the manifold balloon accumulator.

This concept of having a series of constant speed blowers which link to the air manifold through one-way valves, which can be turned on or off, in which the flow capacity of each blower in the sequence is double the capacity of the next lower capacity blower in the sequence, allows adjustment of the total amount of air compressed through the filter and humidity/temperature adjustment device into the manifold by multiple levels. If 5 blowers, each one with twice the capacity of the next smaller blower, in which the smallest blower is sized to provide enough air for one patient, then flow rate into the air manifold can be adjusted by 31 evenly spaced flow rate steps to correspond to nearly the volume of air actually being inhaled by all of the patients on the manifold for up to 31 patients. Such a geometric progression of blower fan capacities can be used to match the total air input into the air manifold to what the patients need without having to have variable speed controls on the blowers or dumping most of the compressed air through the controllable exhaust valve in order to maintain accurate pressure control in the manifold.

Supply of pressurized air to the air manifold can alternatively be by means of a variable speed drive for a single variable output blower, and in that case, air manifold pressure can also be controlled to maintain an accurate target pressure in the air manifold, without the controlled leakage valve shown in FIG. 1. However, this approach has no built-in redundancy in case of a failure of the variable speed drive or the blower. Especially in poor countries, it is more desirable to vary the pressurized air input by the number of blowers turned on and to make the blowers be single speed blowers similar to the type used in commercially available vacuum cleaners.

The air manifold pressure should be nearly equal to the oxygen manifold pressure. The air and oxygen in the manifolds both should have their humidity and temperature adjusted before entering the manifolds, as is the case in Example 1 and FIG. 1. The exhaust gas from exhalation normally vents directly into the hospital ward beside the patient through a variable pressure relief valve and then through a virus-capable filter, as shown in FIG. 1, but it may also exit the hospital ward through an exhaust manifold (not shown in FIG. 1).

The control system receives inputs from pressure sensors, humidity sensors and optimally oxygen sensors and/or blood oxygenation data from pulse oximeters. The control system triggers the opening and closing of solenoid valves, turning blower fans on and off, and can trigger various alarms. The details of this control system are not shown but follow conventional design processes.

For this example, both the oxygen and air manifold are set at 2600 pascals above the local atmospheric pressure, which is an appropriate pressure for direct ventilation of most patients. This pressure is measured at the midpoint of the manifolds, right beside the manifold balloon accumulators. This is also the location at which pressurized air and oxygen enter the manifolds. The design criteria are that the maximum pressure drop in these manifolds is no more than 130 pascals, which represents a 5% pressure drop in terms of the manifold pressure above atmospheric pressure. We have calculated that this implies an inside manifold diameter of about 10 cm.

At each patient's bedside, there is a section of tubing between the manifolds and the patient which interfaces with both the air and oxygen manifolds through a filter that prevents contamination of the air or oxygen manifolds by virus, and the exhaust solenoid valve. This section of tubing is referred to herein as the patient manifold. There is a pressure relief valve between the exhaust solenoid and the exhaust which maintains positive PEEP, after which the exhausted gas can be vented into the hospital ward through a filter or other device to make sure the exhaled gases are sterile, or through a shared exhaust manifold. Said pressure relief valve can either be adjusted manually by a nurse or respiratory therapist, or the pressure relief valve may be adjustable through the control system.

It is desirable that the variable pressure relief valve which opens during the exhaust cycle should be controlled versus atmospheric pressure rather than simply the pressure differential between the patient manifold and the exhaust on the other side of the exhaust solenoid.

FIG. 1 shows balloon accumulators on both the air and oxygen manifolds. These are optional features that have the effect of minimizing pressure fluctuations as the solenoids open and close. It is desirable that these manifold balloon accumulators would be at the midpoint of the air and oxygen manifolds.

### Example 2 (Prior Art)

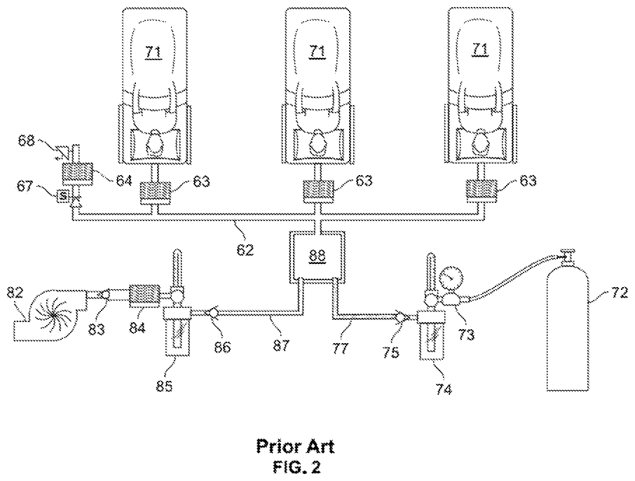

Example 2 and FIG. 2 illustrate by contrast a prior art manifold ventilator. In this case, all patients served by the gas manifold must have the same oxygen concentration, inhalation pressure, humidity, and breathing cycle.

### Example 3

Example 3 and FIG. 3 describe an alternative layout for the patient interface. In this example, there is a balloon accumulator beside each patient. Oxygen and air are introduced sequentially into this balloon accumulator so as to maintain a target pressure and a target oxygen concentration. In this case, the air and oxygen manifolds are set at 5000 pascals above the local atmospheric pressure, which is low enough that it is impossible to burst the balloon accumulators with the air and oxygen manifold pressure. There is a pressure sensor that measures the pressure in each bedside balloon accumulator. There may also be an oxygen sensor located inside the balloon accumulator (not shown in FIG. 3). FIG. 3 also shows a bedside module that is capable of controlling the solenoid operations for that particular patient even if the master control system is offline. The solenoid valves feeding air and oxygen into the balloon accumulator are opened and closed to control the pressure and oxygen concentration. One part of the target is the pressure, and the other part could either be oxygen concentration in the balloon itself, measured directly with an oxygen sensor, or blood oxygenation measured by a pulse oximeter worn by the patient.

FIG. 3 shows a bedside pressure relief valve for the exhaust. This design enables individualized PEEP, and the exhaust may either go to the hospital ward atmosphere through a virus-capable filter as is shown or alternatively the exhaust can go to an exhaust manifold. It is highly desirable that this valve opens according to the differential pressure between the patient manifold and the local atmospheric pressure.

### Example 4

Example 4 is illustrated by FIG. 4, which shows a design in which the manifolds loop around the hospital ward. There are valves between each next neighbor set of patients served by the manifolds. This enables having different manifold pressures in different parts of the hospital ward, and also makes it simpler to provide redundancy in both the air and oxygen manifolds. This also simplifies maintenance.

The manifolds of FIG. 4 form a complete loop, with both oxygen and air points of introduction in two different places around the loops of the manifold pipes, enabling more than one feeder into a loop-shaped manifold, to create a safe backup situation. This may also allow for maintenance or modification of the manifold loops through the capability of closing valves between next neighbor patient beds. Placing valves in the manifold pipes between each pair of next neighbor patients also enables creating different zones with different pressures, while still enabling a single pressure manifold in case of an emergency.

### INDUSTRIAL APPLICABILITY

The COVID-19 pandemic experienced in 2019-2021 has demonstrated the need for a ventilation system that can simultaneously serve multiple patients. In hospitals situated in virus outbreaks, single-patient ventilation systems were quickly fully occupied. Treating additional patients required the procurement of additional ventilators. The capacity of the described invention to supply breathing gas can save lives. The system has the ability to treat a dozen or more patients simultaneously. If more patients need treatment an existing implementation of the invention can be expanded further with a few parts rather than the procurement of a new device. The industrial applicability of the invention is clear.

1. A manifold ventilator capable of servicing multiple patients comprising,
at least one breathing gas supply manifold;
a plurality of lengths of gas exchange tubing;
at least two solenoid valves; and
at least two breathing tubes;
Wherein each solenoid valve has a gas inlet connection and a gas outlet connection; and
Wherein for at least two solenoid valves, the gas inlet connection is connected to the breathing gas supply manifold by a length of gas exchange tubing and the gas outlet connection is connected to a breathing tube;
Whereby when at least one of the solenoid valves is in the open position there is an isolated passageway for breathing gas from the breathing gas supply manifold through the open solenoid to the breathing tube.

2. The manifold ventilator of claim 1, wherein the ventilator comprises at least two breathing gas supply manifolds, wherein the breathing gas of at least two breathing gas supply manifolds can be mixed prior to the entry of the gases into at least one breathing tube.

3. The manifold ventilator of claim 2, wherein at least one breathing gas supply manifold supplies a breathing gas that is at least 50% oxygen.

4. The manifold ventilator of claim 2, wherein at least one breathing gas supply manifold supplies a breathing gas that is at least 50% nitrogen.

5. The manifold ventilator of claim 1, wherein the ventilator comprises at least one exhaust manifold capable of maintaining a positive end expiratory pressure.

6. The manifold ventilator of claim 1, wherein the ventilator comprises at least two elastomeric balloon pressure accumulators attached to at least two breathing tubes.

7. The manifold ventilator of claim 1, wherein the ventilator comprises at least one balloon accumulator attached to at least one of the breathing gas supply manifolds.

8. The manifold ventilator of claim 5, wherein the ventilator comprises at least one vacuum accumulator connected to the exhaust manifold.

9. The manifold ventilator of claim 1, wherein the ventilator comprises at least one sensor positioned on the air manifold; said sensor being electronically linked to a control system for at least one solenoid valve to exhaust air from the air manifold in order to accurately maintain a target pressure therein.

10. The manifold ventilator of claim 6 in which a pressure sensor senses the pressure in one or more bedside balloon accumulators so as to control the inhalation pressure for that individual patient.

11. The manifold ventilator of claim 6, wherein an oxygen sensor is linked to a bedside balloon accumulator and provides oxygen concentration data to the control system which is used to control the opening time of an oxygen solenoid and linking the bedside balloon accumulator to the patient manifold.

12. The manifold ventilator of claim 2, wherein data from a pulse oximeter is used to adjust the opening time of an oxygen solenoid and an air solenoid providing breathing gas to a patient.

13. The manifold ventilator of claim 2, wherein for at least one patient receiving a breathing gas, the solenoid valve in line with the oxygen-containing manifold is opened and closed before the solenoid valve in line with the nitrogen-containing manifold is opened and closed, in which the oxygen going to the patient is controlled by the relative open time of the solenoid valve in line with the oxygen-containing manifold compared to be open time of the solenoid valve in line with the nitrogen-containing manifold.

14. The manifold ventilator of claim 13 in which the relative opening time of the solenoid valve in line with the oxygen-containing manifold compared to the opening time of the solenoid valve in line with the nitrogen-containing manifold is controlled to achieve a target oxygenation levels measured by a sensor worn by the patient.

15. The manifold ventilator of claim 2 wherein breathing gas is supplied to three or more patients.

16. The manifold ventilator of claim 6, wherein breathing gas is supplied to at least one patient that has been diagnosed as infected with the COVID-19 virus.

17. The manifold ventilator of claim 16, wherein breathing gas is simultaneously supplied to at least three patients that have been diagnosed as infected with the COVID-19 virus.

18. The manifold ventilator of claim 10 breathing gas is simultaneously supplied to at least three patients The manifold ventilator of claim 18, wherein at least two patients receive a breathing gas with a different level of oxygen concentration.

20. The method of treating at least one patient diagnosed with respiratory distress symptoms related to COVID-19 infections comprising:
(a) Establishing via a clinical test the patient has received a positive indication for COVID-19 exposure;
(b) Diagnosing via medical examination that the patient is in respiratory distress;
(c) Administering an oxygenated-breathing gas through a shared manifold ventilator, wherein the ventilator comprises:
at least one breathing gas supply manifold;
a plurality of lengths of gas exchange tubing;
at least two solenoid valves; and
at least two breathing tubes;
Wherein each solenoid valve has a gas inlet connection and a gas outlet connection; and
Wherein for at least two solenoid valves, the gas inlet connection is connected to the breathing gas supply manifold by a length of gas exchange tubing and the gas outlet connection is connected to a breathing tube;
Whereby when at least one of the solenoid valves is in the open position there is an isolated passageway for breathing gas from the breathing gas supply manifold through the open solenoid to the breathing tube.
